(19) United States
(12) Patent Application Publication
(10) Pub. No.: US 2006/0052304 A1
(43) Pub. Date: Mar. 9, 2006

(54) Method for remodeling bone and related sutures

(75) Inventor: Dennis R. Stewart, Los Gatos, CA (US)

Correspondence Address: ROBINS & PASTERNAK, 1731 EMBARCADERO ROAD, SUITE 230, PALO ALTO, CA, 94303, US

(73) Assignee: BAS Medical, Inc., San Mateo, CA (US)

(21) Appl. No.: 11/191,824

(22) Filed: Jul. 27, 2005

Related U.S. Application Data
(60) Provisional application No. 60/619,509, filed on Oct. 13, 2004.
(60) Provisional application No. 60/607,353, filed on Sep. 2, 2004.

(51) Int. Cl. A61K 38/17; A61K 38/22

(52) U.S. Cl. 514/12

(57) ABSTRACT

The invention relates to the discovery that relaxin receptors exist in bone and related sutures. As such, bone can be remodeled, repaired, removed or grown. Particularly, the invention pertains to a method for modifying a target bone by administering a relaxin compound which binds to relaxin receptors and by monitoring a change in the target bone. The invention further encompasses methods of modifying the height of a human subject.

### FIELD OF THE INVENTION

The invention relates to the discovery that relaxin receptors exist in bone and related sutures. As such, bone can be remodeled, repaired, removed or grown. Particularly, the invention pertains to a method for modifying a target bone by administering a relaxin compound which binds to relaxin receptors and by monitoring a change in the target bone. The invention further encompasses methods of modifying the height of a human subject.

### BACKGROUND OF THE INVENTION

Bone is vibrant, living tissue that constantly regenerates. Bone tissue makes up the body's skeleton, providing shape and support for the body, as well as protection for some organs. Bone is strong, yet also light and flexible. Its two main ingredients, calcium phosphate and calcium carbonate, provide strength and rigidity. The arrangement of its fibers, and the fact that bones are connected by pliant muscles and joints, enable movement and flexibility. Depending on their level of activity, bones gain mass and change shape. Bone also serves as a storage site for minerals and provides the bone marrow for the development and storage of blood cells. There are three types of bone tissue such as compact tissue (i.e., the harder, outer tissue of bones), cancellous tissue (i.e., the sponge-like tissue inside bones), and subchondral tissue (i.e., the smooth tissue at the ends of bones, which is covered with another type of tissue called cartilage). The combination of compact and cancellous tissue is called the periosteum. Beneath the hard outer shell of the periosteum, blood and lymphatic vessels run through tunnels and canals to carry nourishment to the bone. Muscles, ligaments, and tendons may attach to the periosteum.

Bones are classified by their shape as long, short, flat, and irregular bones, primarily, they are referred to as long or short bones. There are about 206 bones in the human skeleton, not including teeth and sesamoid bones (i.e., small bones found within cartilage). These bones are the 80 axial bones, which include the head, facial, hyoid, auditory, trunk, ribs, and sternum; and the 126 appendicular bones, which include arms, shoulders, wrists, hands, legs, hips, ankles, and feet.

There are three different types of bone cells. Osteoblasts are found within the bone and their function is to form the tissue and minerals that give bone its strength. Osteoclasts are large cells that are formed in the bone marrow and their function is to absorb and remove unwanted tissue. Thus, osteoblasts and osteoclasts maintain a delicate equilibrium in the bone, forming bone and removing bone throughout the life cycle of a living organism. Osteocytes are also found within the bone and their function is to help maintain bone as living tissue. In addition, adipose (fat) cells and hematopoietic cells are found within the bone marrow, wherein hematopoietic cells are responsible for producing blood cells.

Relaxin has generally been known as pregnancy hormone, where it plays an important role during parturition, growth and quiescence of the uterus, growth and development of the mammary gland, and regulation of cardiovascular function. Interestingly, binding sites for relaxin were found in reproductive tissue, brain, and heart but the nature of the relaxin receptor remained elusive. Relaxin belongs to the family of peptide hormones that also includes insulin and insulin-like growth factor (IGF). Hsu et al. showed that two G-protein-coupled receptors, LGR7 and LGR8, are capable of mediating the action of relaxin through an adenosine 3′,5′-monophosphate (cAMP)-dependent pathway that is distinct from that of the structurally related insulin and insulin-like growth factor ligands. When antepartum mice were treated with a soluble ligand-binding region of the LGR7 receptor, it caused parturition delay. Hsu et al. speculated that studies on relaxin receptors could allow the design of relaxin analogs (agonists and antagonists) for the treatment of disorders of labor onset (Hsu et al. (2002) Science 295:671-574).

Relaxin is also known to be involved in endometrial differentiation during embryo implantation, in infarcts and wound healing, in ameliorating fibrosis, and possibly tumor growth and progression. Ivell et al. describe the various roles of relaxin, including two closely related peptide hormones, one specific for the brain, and another involved in testicular descent and ovarian apoptosis. The crystal structure of relaxin is similar to that of insulin, wherein the alpha helices of the A- and B-chains support the conformation of each other, and are held together by two interchain cysteine bridges and one intrachain bridge in the A-chain. Like insulin, mature relaxin appears to the result of post-translational processing. The function of relaxin, in rodents and carnivores, is to prepare the birth canal for parturition, a special endocrine function. However, this is not the case in other species, for example, cows, sheep and humans. The reduced importance of relaxin in peripartum physiology suggest other possible roles for relaxin which have so far gone unnoticed, including a function in cardiovascular physiology and cancer (Ivell et al. (2002) TRENDS in Endocrinology & Metabolism, Review, Vol. 13(8):343-348).

Bathgate et al. discuss potential new roles for relaxin as neuromodulators in the central nervous system (CNS) and as regulators of normal collagen turnover. They describe a new gene encoding relaxin-3 (RLN-3) that appears to be mostly expressed in the brain. They also discuss a role for relaxin-1 (RLN-1) in regulating the normal turnover of collagen besides its established function in pregnancy and parturition. Mapping studies indicate a developed network of relaxin-3, relaxin-1 and relaxin receptor-expressing cells in the brain, suggesting that relaxin peptides may play a role in the central nervous system (Bathgate et al. (2003) TRENDS in Endocrinology and Metabolism (Review) 14(5):207-213). Hence, the biological significant of the relaxin peptide family is expanding, as are their potential clinical uses.

Due to the complexities of a bone's function, from providing strength and support for the body, to serving as a site for development and storage of blood cells, many disorders and diseases can affect bone. What is needed in the art are methods of modifying bone, including repairing and remodeling diseased or inadequate bone. The present invention satisfies these needs.

### BRIEF SUMMARY OF THE INVENTION

The present invention relates to the discovery that relaxin receptors exist in bone and related sutures and that bone can be modified via these relaxin receptors. One advantage of the invention is that a target bone can be modified by administering a relaxin compound that binds to the relaxin receptors and by monitoring a change in the target bone. This method is much less invasive than currently known methods that are used to treat bone disorders and it does not lead to serious adverse side effects as is commonly the case with known medications and/or drugs. Another advantage of the present invention is that the target bone may be remodeled, repaired, removed or grown depending on the type and need of the bone. For example, a bone fracture may be repaired by administering relaxin. Similarly, a bone may be grown in size or height by administering relaxin. As such, the instant invention allows for various treatments of bone such as modifications and repair that may otherwise not be achievable. In addition, the present invention allows for treatments of bone that may otherwise only be achievable through the use of invasive procedures and/or the administration of a combination of drugs leading to a host of adverse side effects. Thus, another major advantage of the present invention is that the methods described herein provide a more natural and less invasive form of treating a variety of bone disorders and/or bone malfunctions.

One aspect of the invention provides a method for modifying a target bone in a mammalian subject, wherein the method comprises (i) administering a relaxin compound to the subject wherein the compound binds to a relaxin receptor in the target bone; and (ii) monitoring a change in the target bone, wherein the change results in a modified target bone including, but not limited to, remodeled bone, repaired bone, bone grown in size and bone grown in length. As such, the change in the target bone can include a change in bone growth, bone strength, bone movement, bone location, bone removal, bone deposition and bone fracture repair. Overall, the target bone (e.g., any bone that comprises osteoblasts and osteoclasts) can be modified in various ways which includes growing, remodeling and repairing the bone. For example, a repaired bone can include a bone fracture, a bone malformation, a bone tumor, or the like. The method of the instant invention applies to mammalian subjects such as humans, dogs, cats, rats, mice, horses and others.

Another aspect of the invention provides for the administration of a relaxin compound to a target bone such that the compound binds to a relaxin receptor in the target bone thereby affecting (e.g., modifying) the target bone. Relaxin receptors of the instant invention include, but are not limited to, LGR7, LGR8, GPCR135 and GPCR142. The relaxin compounds include, but are not limited to, H1 relaxin, H2 relaxin, H3 relaxin, relaxin-like factor (RLF) and combinations thereof. For example, H1 relaxin can bind to a relaxin receptor such as LGR7 and LGR8; H2 relaxin can bind to a relaxin receptor such as LGR7 and LGR8; and H3 relaxin can bind to a relaxin receptor such as GPCR135 and GPCR142. The relaxin compound can be a relaxin analog that functions as a receptor agonist, wherein the receptor agonist may be a compound that binds to a relaxin receptor in the target bone and activates relaxin receptor activity. Alternatively, the relaxin compound can be a relaxin analog that functions as a receptor antagonist, wherein the receptor antagonist may be a compound that binds to a relaxin receptor in the target bone and blocks relaxin receptor activity. Generally, a relaxin receptor is found in bone tissue including, but not limited to, osteoblasts, osteoclasts, osteocytes, odontoblasts and chondrocytes. Furthermore, a relaxin receptor can be found in bone marrow tissue including, but not limited to, mature myeloid elements of erythrocytes and mature myeloid elements of leukocytes. For example, a relaxin receptor can be found in bone marrow tissue of immature myeloid elements that are composed of hemocytoblasts, erythroblasts, myelocytes and megakaryocytes. In addition, a relaxin receptor can be found in the sutures between bones. Particularly, a relaxin receptor can be found in the palatal and other sutural ligaments.

The invention further contemplates a method for modifying height in a human subject, wherein the method comprises (i) administering a relaxin compound to the human subject wherein the compound binds to a relaxin receptor in a target bone; and (ii) monitoring the subject for a change in height, wherein the target bone includes, but is not limited to, long bones of the limbs, skeletal bones and vertebral bones. As such, the height of the human subject may be increased between about 0.2 cm to about 30 cm or more. Although, the skilled artisan would understand that any height increase as a result of relaxin administration as discussed herein is within the scope of this invention. The human subject may be between 1 and 30 years of age. Preferably, the human subject is between about 8 and about 18 years of age. The human subject may be a patient suffering from one or more of a variety of conditions such as delayed growth, arthritis, Paget's disease, avascular necrosis, fibrous dysplasia, osteogenesis imperfecta (01), primary hyperparathyroidism, growth hormone deficiency (GHD), growth hormone insensitivity syndrome (GHIS), osteoporosis or the like.

These and other objects, advantages and embodiments of the present invention will be apparent when read with the detailed description and figures which follow.

### BRIEF DESCRIPTION OF THE DRAWINGS

The present invention is best understood when read in conjunction with the accompanying figures which serve to illustrate the preferred embodiments. It is understood, however, that the invention is not limited to the specific embodiments disclosed in the figures.

FIG. 1 depicts relaxin receptors in the hypertrophic zone of the epiphyseal plate cartilage. The relaxin receptor staining (shown as dark shading) is shown in epiphyseal plate chondrocytes. The figure clearly shows that the heaviest staining for relaxin receptors is localized in the hypertrophic layer of the cells (i.e., chondrocytes). These cells are rapidly dividing and are also storing glycogen.

FIG. 2 provides a magnification of FIG. 1.

FIG. 3 shows that relaxin receptors were found in rat osteocytes, odontoblasts, osteoblasts, and osteoclasts. FIG. 3 depicts alveolar bone under the roots of a tooth that had orthodontic movement toward the right. This caused osteoclasts and osteoblasts to localize on the alveolar bone. The dark staining indicates localization of the relaxin receptor (see arrows).

FIG. 4 shows a low magnification of alveolar bone (left) and a high magnification of the crest of the alveolar bone (right) in dog. Dog oral specimens were stained with the relaxin antibody and several tissues were found to contain binding sites for relaxin. Relaxin receptor binding can be seen in osteocytes in the bone.

FIG. 5 depicts the hard palate of a dog, including the suture which is visible in the hard palate. The arrow indicates the suture.

FIG. 6 shows the hard palate of FIG. 5 with relaxin receptor stains. The arrows point to relaxin receptors in bone and suture.

FIG. 7 shows a magnification of the hard palate of FIG. 6. The relaxin receptor stains are clearly visible in the bone as well as in the sutural ligament.

### DETAILED DESCRIPTION OF THE INVENTION

I. Definitions

The following definitions are set forth to illustrate and define the meaning and scope of the various terms used to describe the present invention.

The term “modifying” as in “modifying a target bone”, means, for the purpose of the specification and claims, an alteration or change in form, character or location. For example, a modified target bone can be any bone that has been changed or altered in form (e.g., bone size, bone length, bone width, bone height, and the like) or character (e.g., cellular make-up of the bone, strength of the bone, and the like) or location (e.g., bone movement, bone removal, bone deposition, and the like).

The term “administering” as in “administering a relaxin compound”, as referred to herein, means to give or apply a compound (e.g., as a remedy). A compound may be administered in a variety of ways including, but not limited to, oral administration, oral transmucosal (i.e., absorbed through the oral mucosa), administration by injection, transdermal administration (e.g., through a skin patch or gel), rectal administration (e.g., suppository), infusion pump (e.g., through a catheter), and others.

The term “relaxin compound” includes, but is not limited to, a relaxin molecule or ligand (i.e., natural or synthetic); a relaxin mimetic or analog (i.e., natural or synthetic); and any pharmaceutical formulation of relaxin that functions according to the teachings of the invention. A relaxin compound may further include a relaxin agonist and a relaxin antagonist. Human relaxin formulations are described in U.S. Pat. Nos. 5,945,402 and 5,451,872. Relaxin analogs, relaxin derivatives, relaxin-like factors and method of uses thereof are described in U.S. Pat. Nos. 6,200,953; 5,811,395 and 5,911,997 as well as patent application Ser. No. 09/846,149. A process for producing relaxin is described in U.S. Pat. No. 5,759,807. A process and compositions for the isolation of human relaxin is described in U.S. Pat. No. 5,464,756. A method of chain combinations for human relaxin or analogs thereof are described in U.S. Pat. No. 4,835,251. All U.S. patents are incorporated herein by reference in their entirety.

The term “receptor” as referred to herein, means a molecule inside or on the surface of a cell that binds to a specific substance and causes a specific physiologic effect in the cell. For example, a relaxin receptor of a bone cell binds to a relaxin molecule or analog thereof (e.g., a synthetic molecule that resembles a naturally occurring molecule) and causes a specific physiologic effect in the bone cell. When a relaxin receptor binds to specific relaxin molecule or analog thereof, the receptor may be activated or blocked. For example, when a relaxin receptor binds to a relaxin molecule or agonist thereof, the receptor can be activated. Alternatively, when the receptor binds to an antagonist of the relaxin molecule, the receptor can be blocked.

The term “suture” refers to an immovable joint, especially between bones such as between the bones of the skull. The suture includes the sutural ligaments (e.g., palatal ligaments and others). However, for the purpose of the invention, a suture is considered a part of the bone. There are different types of sutures including, but not limited to, the suture between the parietal and frontal bones of the skull (coronal suture); the suture between two halves of the frontal bone (frontal suture; usually obliterated by the age of 6); the suture between the two maxillae of the upper jawbone (intermaxillary suture); the suture between the bones of the palate (palatal suture); the suture between the two nasal bones (internasal suture); the suture between the occipital and parietal bones (lamboid suture); the suture between the occipital and the temporal bones (occipitomastoid suture; a continuation of the lamboid suture); the suture between the parietal and the temporal bones (parietomastoid suture); the suture uniting the two parietal bones (interparietal suture, and sagittal suture); and others.

An “agonist” as used herein, is an endogenous or synthetic substance, including a drug or compound, that can interact with a receptor and initiate a physiological or a pharmacological response characteristic of that receptor (e.g., contraction, relaxation, secretion, enzyme activation, and the like).

An “antagonist” as referred to herein, is an endogenous or synthetic substance, including a drug or compound, that opposes the physiological effects of another endogenous or synthetic substance. At the receptor level, it is a chemical entity that opposes the receptor-associated responses normally induced by another bioactive agent (e.g., endogenous or synthetic bioactive agent).

II. Relaxin Receptors and Compounds

The instant invention relates to the discovery that relaxin receptors are found in the bone and that bone can be modified via these relaxin receptors. Relaxin is a small, two-chain protein. It is structurally similar to insulin and its two chains are linked by disulfide bonds. Relaxin has a unique, mixed function receptor-binding region comprising amino acid residues that evolve sequentially from the central portion of the B chain a-helix. Two arginine residues in positions B13 and B17 that project like forefinger and middle finger from the helix provide the electrostatic element opposed by the hydrophobic (thumb) element isoleucine (B20), offset from the arginines by about 40°. The binding intensity of relaxin to its receptor decreases by three orders of magnitude if alanine is substituted for the binding component isoleucine in position B20. A hydrophobic surface appears to exists on the receptor that offers optimal van der Waals' interaction with b-branched hydrophobic amino acids. Overall, the binding site geometry in relaxin is exceptionally clear (see Büllesbach et al. (2000) The Journal of Biological Chemistry 275(45):35276-35280).

There are a number of genes that encode relaxin, such as relaxin-1, relaxin-2 and relaxin-3. The genes are found in different species and encode mouse, rat, pig and human relaxin proteins. Human relaxin proteins are referred to as human relaxin 1 (H1), human relaxin 2 (H2) and human relaxin 3 (H3). Relaxin-1 encodes a protein (H1 or RNL1) that is primarily expressed in the ovary and placenta of mouse and rat as well as in the decidua, trophoblast and prostate in humans (see Hansell et al. (1991) J. Clin. Endocrinol. Metab. 72(4):899-904). Notably, there is no equivalent of H1 in species below higher primates. Relaxin-2 encodes a protein (H2 or RNL2) that is mainly expressed in the ovary of humans as the major stored and circulating form of relaxin as well as in decidua, trophoblast and prostate (Garibay-Tupas et al. (2000) J. Mol. Endocrinol. 24(2):241-252). H2 is the equivalent of relaxin 1 in other species (e.g., mouse relaxin 1, rat relaxin 1). Relaxin-3 encodes a protein (H3 or RNL3) that is mainly expressed in the brain of rat and mice (e.g., mouse relaxin 3, rat relaxin 3) (see Bathgate et al. (2003), supra) but is also found in the brain of humans (see Bathgate et al. (2002) J. Biol. Chem. 277(2):1148-1157).

Relaxin receptors are leucine-rich repeat containing G-protein-coupled receptors (LGR) (see Hsu et al. (2002) Science 295:671-674). Relaxin activates two orphan G-protein-coupled receptors, LGR7 and LGR8. LGR7 and LGR8 are similar in structure and are related to gonadotropin receptors. They are leucine-rich G-protein-coupled transmembrane receptors. LGR7 is expressed in brain, kidney, testis, placenta, uterus, ovary, adrenal, prostate, skin and heart. LGR7 binds pig relaxin, H2 and H3. Moreover, LGR7 binds both H1 and H3 with high affinity. LGR8 is expressed in brain, kidney, muscle, testis, thyroid, uterus, peripheral blood cells, and bone marrow. LGR8 binds relaxin-like factor (RLF), also referred to insulin 3 (INSL3), H2 and pig relaxin. Relaxin binding to the LGR7 and LGR8 receptors is known to stimulate cAMP pathways (see Hsu et al., supra).

H3 activates two additional G-protein-coupled receptors, GPCR135 and GPCR142. GPCR135 and GPCR142 are also leucine-rich G-protein-coupled receptors but have homology with angiotensin peptide receptors. GPCR135 is expressed in the central nervous system and appears to inhibit cAMP while LGR7 stimulates cAMP. GPCR142 is also expressed in the brain and has a high homology with GPCR135. GPCR135 and GPCR142 bind only H3.

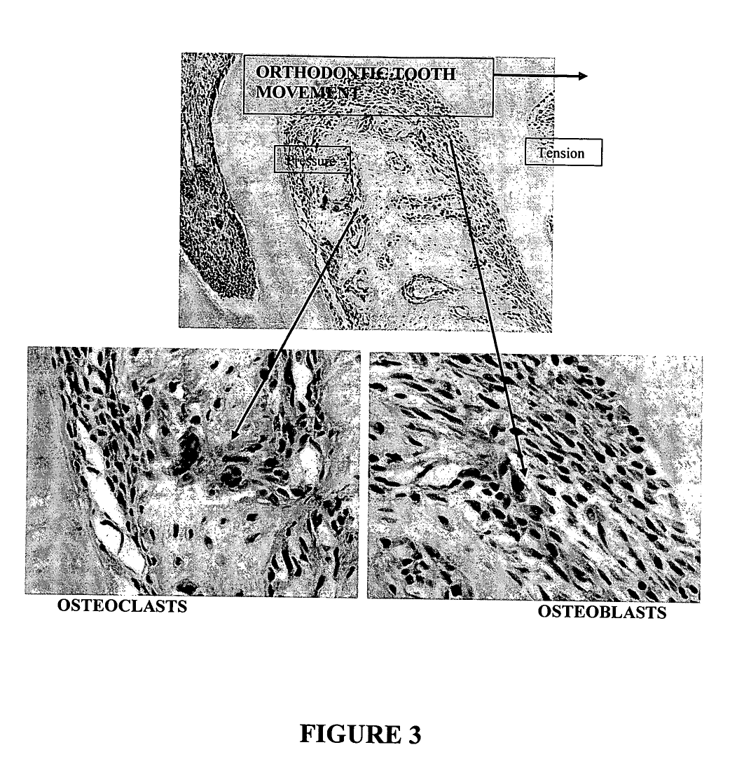

The instant invention pertains to the discovery that the relaxin receptors LGR7 and LGR8 are found in bone. Particularly, these receptors are found in bone tissue including osteoblasts, osteoclasts, osteocytes, odontoblasts and chondrocytes. The skilled artisan will appreciate that other relaxin receptors and binding sites are within the scope of this invention. In one embodiment, the relaxin receptors found in osteoclasts are targeted by administering a relaxin compound such that bone removal (or bone movement) is achieved. In certain embodiments, the relaxin receptors found in osteoblasts are targeted by administering a relaxin compound such that bone deposition or increased bone density is achieved. In yet another embodiment, both relaxin receptors, osteoblasts and osteoclasts, are targeted by administering a relaxin compound such that bone movement or bone remodeling occurs. For example, in the case of a bone fracture, osteoblasts usually predominate. Thus, osteoblasts can be targeted in order to repair or rebuild the fractured bone. In another example, such as for orthodontics, both osteoblasts and osteoclasts are usually present, i.e., osteoblasts on the back side of the tooth filling in the void and osteoclasts on the leading edge of the tooth, breaking down bone to allow movement (see FIG. 3). Thus, osteoblasts and osteoclasts may be targeted simultaneously to achieve bone movement.

The instant invention contemplates the use of relaxin molecules and compounds including, but not limited to, H1, H2, H3, RLF and combinations thereof. Human Relaxin H1 is described in U.S. Pat. Nos. 5,145,962; 5,320,953; 4,871,670; and 5,053,488 which are incorporated herein by reference in their entirety. In a preferred embodiment, the instant invention contemplates the use of H2 relaxin molecules and compounds. Human Relaxin H2 is described in U.S. Pat. Nos. 5,179,195; 4,758,516; and 5,326,694 which are incorporated herein by reference in their entirety. In another preferred embodiment, an H2 relaxin molecule or compound binds to the relaxin receptors LGR7 and LGR8. In another embodiment, an H1 relaxin molecule or compound binds to the relaxin receptors LGR7 and LGR8. In still another embodiment, an H3 relaxin molecule or compound binds to the relaxin receptors GPCR135 and GPCR142. In another embodiment, an RLF molecule or compound binds to the relaxin receptor LGR8.

As indicated above, The LGR7 and LGR8 receptors are part of a family of G-protein-coupled receptors. In general, these receptors stimulate cAMP upon relaxin binding. In fact, relaxin stimulates cAMP in many different cell types such as fibroblasts, uterine cells and THP1 cells (monocytes). Similarly, relaxin can stimulate cAMP in bone tissue and in chondrocytes. cAMP can activate mitogen-activated protein kinase (MAPK) signaling cascades leading to changes in bone tissue and chondrocytes. Specifically, relaxin has been shown to go through the extracellular-signal-regulated kinases 1 and 2 (ERK 1/2) pathway in some tissues, leading to growth, differentiation and development. For example, Dschietzig et al. studied relaxin in vasoconstriction and concluded that relaxin promotes endothelial and epithelial but not vascular smooth muscle via a Ras-independent Raf-1-MEK-1-ERK-1/2 kinase cascade (Dschietzig et al. (2003) Circulation Research 92:32-40). Zhang et al. showed that relaxin treatment of human endometrial stromal cells resulted in rapid activation of p42/44 mitogen-activated protein (MAP) kinase, as well as of MAPK (or ERK) kinase (MEK). Relaxin treatment also induces MAP kinase activation in THP-1 monocytic cells and in human smooth muscle cells, indicating that it may be a major signaling transducer utilized by the relaxin receptor (Zhang et al. (2002) J. Cell. Biochem. 85(3):536-44). Similarly, relaxin can go through the ERK 1/2 pathway resulting in changes in bone tissue and chondrocytes, including growth, differentiation and development in these areas allowing for bone remodeling and bone repair.

Relaxin also effects protein expression. For example, in fibroblasts relaxin stimulates proteins such as collagenases. Unemori et al. showed that relaxin can cause significant collagen turnover both by stimulating collagenase expression and by down-modulating collagen synthesis and secretion (Unemori et al. (1990) J. Biol. Chem. 25; 265(18):10681-10685). Samuel et al. studied relaxin in cardiac fibrosis and concluded that relaxin regulates fibroblast proliferation, differentiation, and collagen deposition and may have therapeutic potential in diseased states characterized by cardiac fibrosis (Samuel et al. (2004) Endocrinology 145(9):4125-4133). Relaxin receptors are found on osteoblasts, thus, relaxin can stimulate bone formation and/or calcium deposition. Relaxin receptors are also found on osteoclasts, thus, releaxin can cause the secretion of enzymes to break down bone. In chondrocytes, the relaxin receptors are mostly localized in the hypertrophic zone where cells are rapidly dividing and storing glycogen. As a result, relaxin binding can cause the chondrocytes to grow larger. Relaxin can also aid in the initiation of development of chondrocytes, and can further aid in the division of bone cells and the production of bone.

Furthermore, blood forming cells (myeloid) are found in bone marrow and a relaxin receptor can be found in bone marrow tissue including, but not limited to, mature myeloid elements of erythrocytes and mature myeloid elements of leukocytes. For example, a relaxin receptor can be found in bone marrow tissue of immature myeloid elements that are composed of hemocytoblasts, erythroblasts, myelocytes and megakaryocytes. Thus, a relaxin receptor found in bone marrow can stimulate red blood cell formation and cells of the immune system upon relaxin binding.

III. General Diagnostic Procedures for Bone Disorders

Diagnostic procedures for bone disorders may include a complete medical history; a physical examination; various laboratory tests for blood, urine, and other body fluids; x-rays; computed tomography (CT or CAT) scan; and others.

i) CT or CAT Scan

A CT or CAT scan is a type of x-ray image that produces cross-sectional views of specific parts of the body. The x-ray beam rotates around the patient, who has been injected with a special dye to help the area of the body reflect the x-ray beam. Information is sent to a computer, which produces the image on a computer screen. A CT scan provides information about bone, muscle and fat. It is also used to assist the physician in locating the exact area for a biopsy.
ii) Magnetic Resonance Imaging (MRI)

An MRI is a scanning procedure that uses radio waves and a magnet to produce cross-sectional views of specific parts of the body such as bone. A computer receives and translates the radio waves into detailed images of the area of the bone under study. MRI scans provide detailed information about soft tissue, the bone marrow cavity, and bone tumors.

iii) Bone Densitometry

Bone densitometry is a noninvasive evaluation procedure that uses x-rays to measure bone mass, or the weight of the skeleton. The amount of bone in the skeleton determines how strong it is. Bone densitometry is often used to measure bone mass in the spine, hips, and arms since these are the areas most likely to fracture when bone mass is low. Bone densitometry is used in combination with other procedures, along with personal and family medical history, to provide information toward or to support a diagnosis.

iv) Radionuclide Bone Scan

A radionuclide bone scan is a nuclear imaging technique that uses a radioactive material such as technetium-99. The radioactive material is injected into the patient's bloodstream to be detected by a scanner. This test shows blood flow to the bone and cell activity within the bone. The tumor absorbs the material, and a camera is used to produce an image using a computer. The bone scan is used to pinpoint the location of the bone tumor, as well as to detect the spread into other organs.

v) Biopsy

A biopsy is a procedure in which a sample of tissue or bone (e.g., bone marrow) is removed from a patient and sent to the pathology laboratory to be examined by a pathologist. There are generally two types of biopsies employed such as needle biopsy and open biopsy. Needle biopsy uses a hollow needle to draw the tissue from the tumor; while open biopsy is a surgical procedure in which an incision is made through the skin to expose the tumor and allow a sample of tissue to be cut or scraped away.

IV. Disorders of the Bone

This invention pertains to the discovery that relaxin can bind to relaxin receptors in the bone and thereby modify bone such as remodel bone, repair bone, grow bone, or remove bone. Thus, the methods of the invention are effective for ameliorating bone diseases and disorders that are associated with bone dysfunction. Since the conditions of the various bone diseases and disorders can be associated with or caused by a variety of disease processes, the methods of the invention are also used to provide further insights into the workings of these disease processes. Bone diseases and disorders that can be treated by employing the methods of the instant invention include, but are not limited to, delayed growth, arthritis, Paget's disease, avascular necrosis, fibrous dysplasia, osteogenesis imperfecta (01), primary hyperparathyroidism, growth hormone deficiency (GHD), growth hormone insensitivity syndrome (GHIS), and osteoporosis.

i) Avascular Necrosis

Avascular necrosis (also called osteonecrosis, aseptic necrosis, or ischemic bone necrosis) is a disease that results from the temporary or permanent loss of blood supply to the bone. When blood supply is cut off, the bone tissue dies and the bone collapses. If avascular necrosis occurs near a joint, collapse of the joint surface may occur. Avascular necrosis may occur in any bone, but most commonly occurs in the ends of a long bone. It may affect one bone, several bones at one time, or different bones at different times. Although avascular necrosis may affect both genders and all age groups, it is diagnosed most often in persons at the ages of 30 to 40.

Avascular necrosis may be the result of traumatic causes including, but not limited to, injury, fracture, or damage to blood vessels as well as non-traumatic causes, including but not limited to, long-term use of medications (e.g., corticosteroids) or excessive, long-term use of alcohol. Other risk factors may be Gaucher disease; blood disorders, such as sickle cell anemia; radiation treatments; chemotherapy and pancreatitis. The most common symptoms for avascular necrosis are minimal early joint pain, increased joint pain as bone and joint begin to collapse and limited range of motion due to pain. In addition to a complete medical history and physical examination, diagnostic procedures for avascular necrosis may include imaging procedures, such as x-ray or CT scan, MRI, radionuclide bone scan, biopsy, functional evaluation of bone (i.e., tests, that usually involve surgery, to measure the pressure inside the bone) and the like (supra).

ii) Fibrous Dysplasia

Fibrous dysplasia is a chronic disorder in which bone expands due to abnormal development of fibrous tissue, often resulting in symptoms such as uneven growth of bones, pain, brittle bones and bone deformities. Any bone of the body can be affected. More than one bone can be affected at any one time. Notably, when multiple bones are affected, it is not unusual for all affected bones to be on one side of the body. However, fibrous dysplasia does not spread from one bone to another. The most commonly affected bones include the femur (thighbone), the tibia (shin bone), the ribs, the skull, the facial bones, the humerus (the bone of the upper arm), the pelvis and the vertebrae in the spine (less often). Some patients develop hormonal problems and a condition called McCune-Albright syndrome. McCune-Albright syndrome, another form of fibrous dysplasia, includes different symptoms, such as early onset of puberty and skin spots, called cafe-au-lait spots. Fibrous dysplasia usually occurs in children and young adults, and is found equally between males and females. The exact cause of fibrous dysplasia is not known, but it is believed to be due to a chemical abnormality in the proteins of the bone. It may also be an inherited disorder. The most common symptoms of fibrous dysplasia are a waddling walk, bone pain (as a consequence of the expanding fibrous tissue in the bone), bone deformity, bone fractures and scoliosis. Scoliosis is a lateral, or sideways, curvature and rotation of the back bones (vertebrae), giving the appearance that the person is leaning to one side. In addition to a complete medical history and physical examination, diagnostic procedures for fibrous dysplasia may include an x-ray, a biopsy, a CT scan and blood tests (supra).

iii) Osteogenesis Imperfecta

Osteogenesis imperfecta (OI), also known as brittle-bone disease, is a genetic disorder characterized by bones that break easily without a specific cause. An estimated 20,000 to 50,000 people in the US have this disease. The disorder occurs in one out of 20,000 to one out of 60,000 live births. OI can affect males and females of all races. The cause of OI is believed to be due to a genetic defect that causes imperfectly-formed, or an inadequate amount of bone collagen (a protein found in the connective tissue). In addition to a complete medical history and physical examination, diagnostic procedures for OI may include a skin biopsy to evaluate the amount and structure of collagen. Additional diagnostic tests may include x-ray (supra) and an examination of the ear, nose, and throat (to detect hearing loss). There are four types of OI. The types and their most common symptoms are discussed below:

Type I is most common. The bones fracture easily and the disease can usually be traced through the family. Affected individuals have a near normal stature or are slightly shorter than normal; they may have a blue sclera (the normally white area of the eye ball); dental problems; and hearing loss (beginning in at the ages of 20 and 30). Most of the bone fractures occur before puberty. Occasionally women will have fractures after menopause. Some affected individuals have triangular faces and a tendency toward spinal curvatures.

Type II mostly affects newborn babies and is frequently fatal. It is usually the result of a new gene mutation. Affected individuals have a very small stature with an extremely small chest and underdeveloped lungs.

Type III tends to be isolated within families. Afflicted individuals have a very small stature (e.g., some are only 3 feet tall). Fractures at birth are very common. An early x-ray may reveal healing of in utero fractures. There is often severe early hearing loss, loose joints and poor muscle development in arms and legs. Another symptom is a barrel-shaped rib cage.

Type IV can frequently be traced through the family. Bones fracture easily and mostly before puberty. Afflicted individuals have a normal or near normal colored sclera but usually problems with teeth. Typical are spinal curvatures and loose joints.

iv) Paget's Disease

Paget's disease of the bone is a chronic bone disorder in which bones become enlarged and deformed. Bone may become dense but fragile. This is mainly due to excessive breakdown and deformation of bone. The disease affects both genders and is rarely found in people under the age of 40. It occurs in up to 3 percent of the US population. The exact cause of Paget's disease is unknown, but it is suggested to be due to a slow viral infection of bone and may include a heredity factor. The most common symptoms of Paget's disease are pain in the affected area, bone deformity in the affected area, susceptibility to fractures in the affected area, and headache and hearing loss (if the affected area is the skull). Diagnostic procedures for Paget's disease of the bone may include x-ray, a radionuclide bone scan and blood tests (supra). Specifically, a blood test that involves measuring the alkaline phosphatase levels (an enzyme found throughout the body) in the bone is recommended since any condition of bone growth or an increased activity of bone cells, including Paget's disease, will cause alkaline phosphatase levels to rise.

v) Primary Hyperparathyroidism

Primary hyperparathyroidism is a metabolic disorder in which one (or more) of the parathyroid glands produces too much parathyroid hormone, which can result in the loss of bone tissue. Too much parathyroid hormone causes too much calcium to be released from the bone. Normally, the function of the parathyroid hormone is to keep blood-calcium levels from going too low by releasing calcium from bones, conserving calcium that would be excreted by the kidneys, and increasing calcium absorption from food. When the hormone overacts, the result is a rise in the blood-calcium level. Primary hyperparathyroidism affects 28 out of 100,000 people in the US each year, and is more prevalent in women than in men. When one parathyroid gland becomes enlarged, the condition is called adenoma. When more than one gland becomes enlarged, the condition is called hyperplasia. Both of these conditions are benign (non-cancerous). In some cases, no cause can be identified. Some known causes include benign (non-cancerous) tumors on the parathyroid glands, or enlargement of the glands. The most common symptoms of primary hyperparathyroidism include loss of appetite, increased thirst, frequent urination, lethargy and fatigue, muscle weakness, joint pain, constipation, kidney pain (due to the presence of kidney stones), nausea, vomiting, abdominal pain, memory loss and depression. Alternatively, this disorder may not present symptoms or complications, and is sometimes discovered during a routine blood test as a part of a physical examination. A diagnosis procedure for primary hyperparathyroidism may involve bone densitometry (supra), to determine bone density and to reveal loss of bone tissue. Bone densitometry can also be used to continually monitor the disorder.

vi) Growth Hormone Related Disorders

Sometimes, short stature is due to a growth hormone deficiency. In some cases of growth hormone deficiency (GHD), the transmission is through an autosomal recessive inheritance of mutations in the growth hormone gene. However, few mutations of the growth hormone gene have been found. Growth Hormone Insensitivity Syndrome (GHIS) is another condition that is marked by short stature. GHIS follows an autosomal recessive pattern of inheritance. The growth pattern in children with GHIS is similar to that in children with severe GH deficiency. The body weight and length may be normal or small at birth while the growth velocity becomes impaired during infancy. The heights during childhood are 4 to 12 standard deviations below the mean. As the child ages, severe growth retardation becomes evident.

vii) Osteoprosis

Osteoporosis is caused by decreased bone mass resulting in fragile bones that are more likely to break. Broken bones, also known as fractures, occur typically in the hip, spine, and wrist. Although, any bone can be affected, fractures of the hip and spine are especially dangerous. A hip fracture almost always requires hospitalization and major surgery. It can impair a person's ability to walk unassisted and may cause prolonged or permanent disability or even death. In addition, spinal or vertebral fractures have serious consequences, including loss of height, severe back pain, and deformity. Progressive osteoporosis often causes a stooped posture or a humpback (kyphotic curve) and it commonly affects the thoracic and thoracolumbar regions of the spine leading to debilitating pain. The structural deterioration of bone further increases the risk for fracture in the hip, spine, and wrist.

According to the National Osteoporosis Foundation, osteoporosis is a major public health threat for an estimated 44 million Americans (i.e., about 55 percent of the people 50 years of age and older). Many Americans have low bone mass, which puts them at increased risk of developing osteoporosis and related fractures. 10 million individuals in the U.S. are estimated to have the disease and about 34 million more are estimated to have low bone mass, placing them at increased risk for osteoporosis. Of the 10 million Americans estimated to have osteoporosis, eight million are estimated to be women and two million are estimated to be men. One in two women and one in four men over age 50 will likely have an osteoporosis-related fracture in her or his remaining lifetime.

Osteoporosis can be diagnosed via a physical examination, including a review of the patient's medical and family history as well as laboratory tests including complete blood count, urinalysis, and thyroid function. A neurological evaluation may also be required. It assesses the patient's symptoms including pain, numbness, paresthesias (e.g., tingling), extremity sensation and motor function, muscle spasm, weakness, and bowel/bladder changes. Either a CT Scan or MRI study (supra) may be required if there is evidence of neurological dysfunction. If a fracture is suspected, a x-ray is performed. Furthermore, bone density test may be required to accurately assess the disease and confirm a diagnosis of osteoporosis (supra). For example, DEXA (dual energy x-ray absorptiometry) is a radiographic test used to measure bone density in the spine, hip, and wrist. Bone Mineral Density (BMD) can also be used to measure bone density in the spine, hip, and wrist. This procedure is painless and noninvasive. It may be used to confirm a diagnosis of osteoporosis. BMD also detects low bone density and the rate of bone loss. Low bone density is an indicator, or early warning, that osteoporosis exists.

V. Bone Formation and Treatment of Bone Disorders with Relaxin

Bone is formed in two ways such as by direct mineralization of matrix secreted by osteoblasts (i.e., intramembranous ossification) or by deposition of bone matrix on a pre-existing cartilage matrix (i.e., endochondral ossification). The bone tissue that appears first is primary or woven (in both processes). Primary bone is temporary tissue and is eventually replaced by lamellar or secondary bone. During bone growth, areas of primary bone, areas of resorption, and areas of secondary bone appear side by side. This combination of bone synthesis and removal (i.e., remodeling) occurs in growing bones and throughout adult life. However, the rate of change in adults is considerably slower than in young children and adolescents (see Basic Histology Text & Atlas, Luiz Carlos Junqueira and Jose Carneiro, Lange Medical Books, McGraw-Hill, New York, 2003; Chapter 8, Bone, pages 141-160).

Intramembranous ossification is the source of most flat bones. This process also contributes to the growth of short bones and the thickening of long bones. The process begins when groups of cells differentiate into osteoblasts. Osteoblasts produce bone matrix and calcification follows. This results in the encapsulation of some osteoblasts which then become osteocytes. The islands of developing bone form walls that delineate into long cavities with capillaries, bone marrow cells, and undifferentiated cells. Eventually, the fusion of the walls gives the bone a spongy structure.

Endochondral ossification is principally responsible for the formation of short and long bones. This process includes a sequence of events that leads to primary and secondary ossification centers in bone. During their expansion and remodeling, these centers produce cavities that are gradually filled with bone marrow. In the secondary ossification centers, cartilage remains in two regions, the articular cartilage and the epiphyseal cartilage (or epiphyseal plate). The articular cartilage persists throughout adult life and does not contribute to bone growth or length. However, the epiphyseal cartilage is responsible for the growth and length of the bone. The epiphyseal cartilage disappears in adults, which is why bone growth ceases in adulthood.

The epiphyseal cartilage is divided into five zones: (1) the resting zone; (2) the proliferative zone; (3) the hypertrophic cartilage zone; (4) the calcified cartilage zone; and (5) the ossification zone. The resting zone consists of hyaline cartilage without morphologic changes in the cells. The proliferative zone contains chondrocytes (i.e., mature cartilage cells) that divide rapidly and form columns of stacked cells parallel to the long axis of the bone. The hypertrophic cartilage zone contains large chondrocytes whose cytoplasm has accumulated glycogen and the resorbed matrix is reduced to thin septa between the chondrocytes. The calcified cartilage zone includes dead chondrocytes, and the thin septa of cartilage matrix become calcified by the deposit of hydroxyapatite. Finally, the ossification zone contains endochondral bone tissue, blood capillaries, and osteoprogenitor cells (which form osteoblasts). The osteoblasts then deposit bone matrix over the three dimensional calcified cartilage matrix.

As shown above, growth in length of a long bone occurs by proliferation of chondrocytes in the epiphyseal cartilage (or epiphyseal plate). Bone growth is associated with partial resorption of preformed tissue and the simultaneous laying down of new bone, wherein bone growth exceeds bone loss. This process allows the shape of the bone to be maintained while it grows. The rate of bone remodeling (bone turnover) is very active in young children. Their bone turnover can be 200 times faster than in adults. In adults, bone remodeling is a dynamic process that occurs simultaneously in multiple locations of the skeleton, not related to bone growth.

The treatment or remodeling of bone by administering relaxin compounds can be accomplished in a variety of ways. Relaxin compounds can be administered as relaxin mimetics or analogs (i.e., natural or synthetic), relaxin agonists, relaxin antagonists, and other pharmaceutical formulations of relaxin. For example, treatment with relaxin can aid in recovery of a broken bone, wherein relaxin is administered either locally by injection or systemically through the use of a subcutaneous administration or implant. The implant eluting pump can be localized in the vicinity of the bone fracture. Relaxin stimulates osteoblasts in the fracture area to activate or enhance osteoblast activity. Furthermore, relaxin aids in restoring blood flow to the fracture site by stimulation of angiogenic factors from wound macrophages. In order to stimulate bone growth, relaxin can be supplied to the bone through general systemic administration if growth of many bones is desired. Alternatively, if specific bones are to be targeted, a local application of relaxin may be preferred. Relaxin also stimulates chondrocytes of the growth plate to enhance bone formation. In some instances, treatment with relaxin in order to grow bone can require a long term commitment as the growth process usually occurs over a time period of many months. The skilled artisan will appreciate that both long term and short term treatments with relaxin are within the scope of the invention.

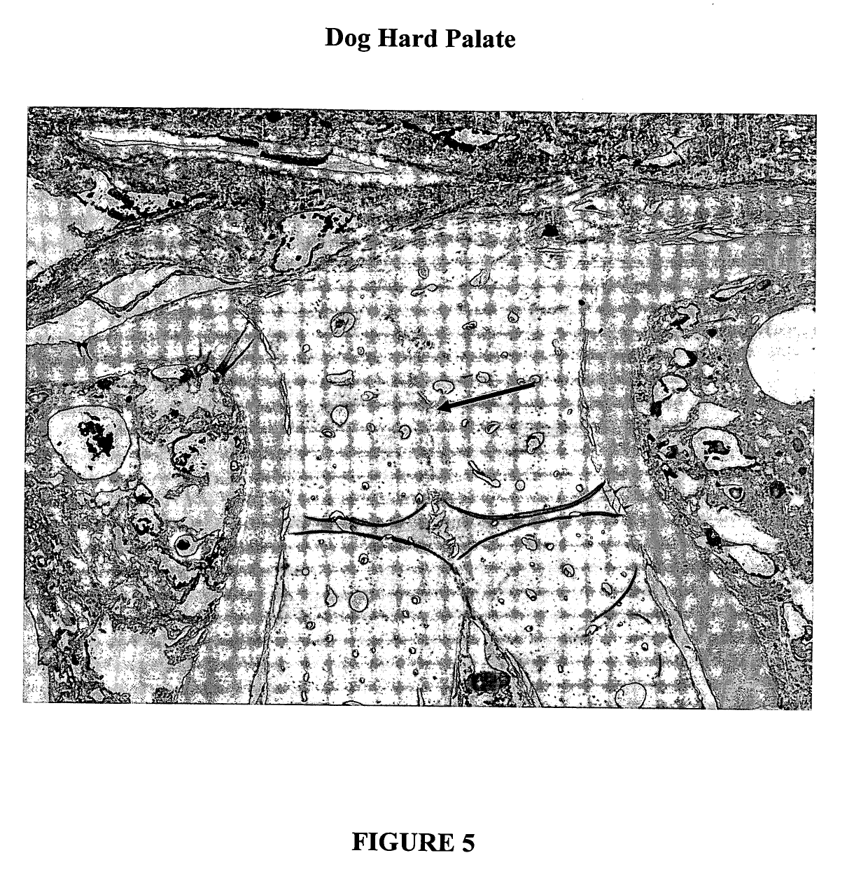

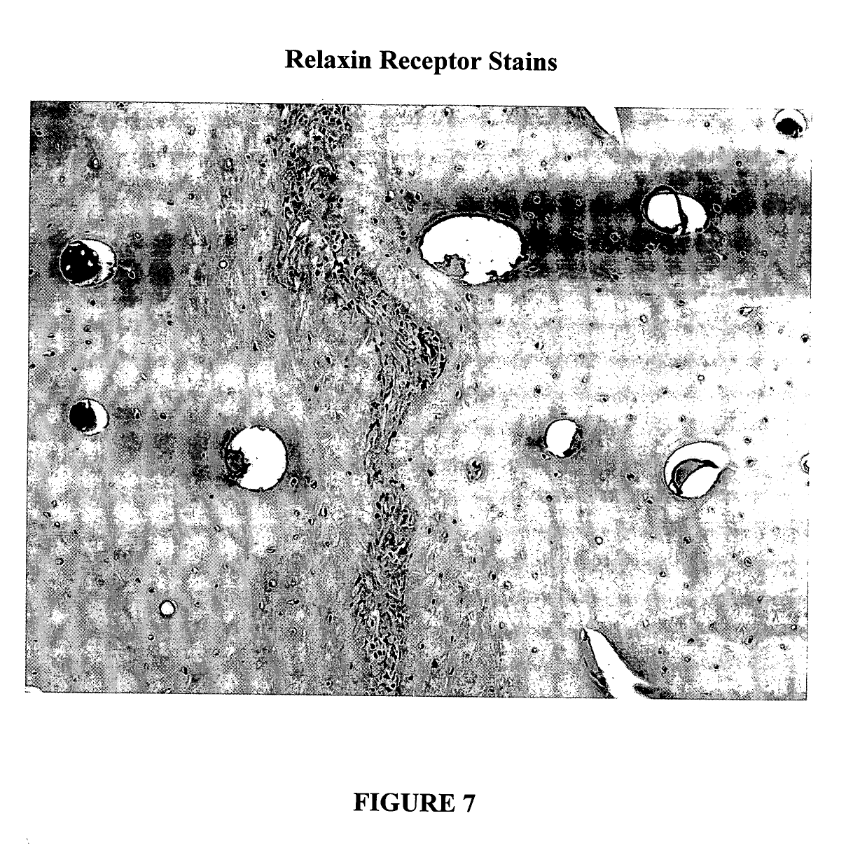

The administration of relaxin can also aid in the expansion of bone. This includes sutural adaptation following orthopedic expansion in growing and non-growing patients. Orthodontic or orthopedic expansion can effectively correct or camouflage a skeletal problem. As shown in the Figures, relaxin receptors are found in bone and sutures (see FIGS. 5, 6 and 7). The figures show that the antibodies to the relaxin receptors bind to cells in the sutural ligament, indicating the presence of relaxin receptors (see FIG. 7). Thus, relaxin can be used to stimulate tissue remodeling of the ligament that holds the two bones of the suture. There is a tremendous need in the art for expanding sutures for various treatments. Relaxin can be applied to effect the suture and sutural ligament such that the bones expand or move in a desired direction to correct for a skeletal defect. For example, sutural expansion is used in orthodontics when the arch must be expanded. This means that the bones that are held together by the ligament must be moved. Relaxin has the ability to soften ligamentous tissue by remodeling collagen. As such, relaxin softens the ligament, allowing the bones to be moved apart faster and with less pain.

Relaxin can also help to prevent the relapse that often occurs following palatal expansion. This technique is known in orthodontics and uses braces. The braces, attached to several maxillary teeth on the left and right sides of the arch are pushed laterally by a jack screw device spanning across the palate. Gradually turning the screw separates the left and right sides of the expansion appliance, transmitting forces to the teeth, which in turn push against the maxillary bone. At the mid-palatal suture, the left and right halves of the maxilla are gradually separated by these forces. The tension on the fibrous suture stimulates bone formation in the stretched web of fibers, which gradually restores the suture to its normal thickness as the stretched fibers are converted into new bone. This technique allows the orthodontist to create a wider maxillary bone and wider maxillary dental arch. In adults, the maxillary palate suture closes and becomes very difficult to separate. Relaxin can extend the age at which palatal expansion can be employed.

Relaxin can be used as adjunctive therapy when sutures of any kind need to be expanded. For example, sutures of cranial bones may need expansion with reconstructive or orthognathic (oral jaw-straightening) surgeries. Relaxin's ability to soften the ligaments attached to these bones makes the separation of the bones much easier. For example, complex orthognathic surgery can involve cuts deep within the facial structures. But with dentofacial orthopedics (D.O.) in combination with relaxin treatment, the changes can be made gradually over a period of weeks, allowing nerves, blood vessels, and other soft tissues to stretch and grow, whereas a sudden and single surgical repositioning may cause damage of delicate soft tissues. This is comparable to the idea of extracting and reimplanting a tooth versus moving it gradually with orthodontics. In the sudden repositioning by extraction, the pulpal nerve and blood vessels, and the periodontal ligament would be severely damaged, whereas with the orthodontic movement in combination with relaxin, these tissues move safely along with the tooth.

Other applications for relaxin include a decrease in scar tissue formation following frenectomies; reduction in soft tissue envelop pull following orthognathic surgery (e.g., reducing the tension of stretched palatal mucosa); enhanced gingival remodeling following space closure of extraction sites, and the like.

A human subject suffering from a bone disease or disorder can be treated with relaxin. The human subject may be a patient suffering from one or more of a variety of conditions or diseases such as arthritis, Paget's disease, avascular necrosis, fibrous dysplasia, osteogenesis imperfecta (OI), primary hyperparathyroidism, growth hormone deficiency (GHD), growth hormone insensitivity syndrome (GHIS), osteoporosis or the like. A patient suffering from a bone disease or bone related condition can be treated with relaxin to alleviate the symptoms of the disease, prevent further deterioration as a result of the disease, help to counteract the negative side effects of the disease, and/or reverse the disease or condition.

VI. Modifying Height through Administration of Relaxin

Short stature is a common condition in all societies. Individuals are usually considered short if their height is in the lowest 2.5 percent of the general population. Short stature can result from many factors, such as nutritional deficiency, chronic illness and disease (supra), medications, genetic abnormalities and multiple inherited factors, disorders of hormones including growth hormone deficiency and insensitivity (supra), degeneration, autoimmune diseases (such as rheumatoid arthritis), viruses, and benign or malignant tumors. Familial short stature is the most common category of short stature and appears genetically related, although there is no identified gene that is solely responsible for familial short stature. Overall, any factors that can affect the integrity of the bones may contribute to short stature. Thus, it is an objective of this invention to provide a remedy for short stature and delayed growth.

The invention encompasses a method for modifying height in a human subject, wherein a relaxin compound is administered to the human subject. The relaxin compound binds to a relaxin receptor in one or more target bones (e.g., skeletal bones) which causes the bones to grow. The growth plate of long bones is where bone elongation occurs. The instant invention demonstrates that chondrocytes in the growth plate contain relaxin receptors. In one embodiment of the invention, relaxin compounds target chondrocytes in the growth plate to enhance bone formation. In another embodiment of the invention, the target bones are the long bones of the limbs. In another embodiment, the target bones are the skeletal bones. In still another embodiment, the target bones are the vertebral bones. The human subject can be monitored for a change in height. As such, the height of the human subject may be increased between about 0.2 to about 30 cm. The human subject is preferably between about 8 and about 18 years of age. Between the ages of 8 and 18, bone is growing actively and is most susceptible to treatment with relaxin. The human subject may suffer from short stature or delayed growth and can be treated with relaxin in order to grow in height.

### VII. EXAMPLES

The following specific examples are intended to illustrate the invention and should not be construed as limiting the scope of the claims.

### Example 1

### Localization of Relaxin Receptors in Bone and Sutures

An antibody to the human relaxin receptor was generated in rabbits and its use has previously been reported (Hsu et al. (2002), supra). This antibody was obtained and used to localize relaxin receptors in rat tissues. In these tissues, relaxin receptors were found in osteocytes, odontoblasts, osteoclasts, and osteoblasts (see FIG. 3). Furthermore, relaxin receptors were found in periodontal ligament (PDL), gingival fibroblasts, and gingival epithelium in rats.

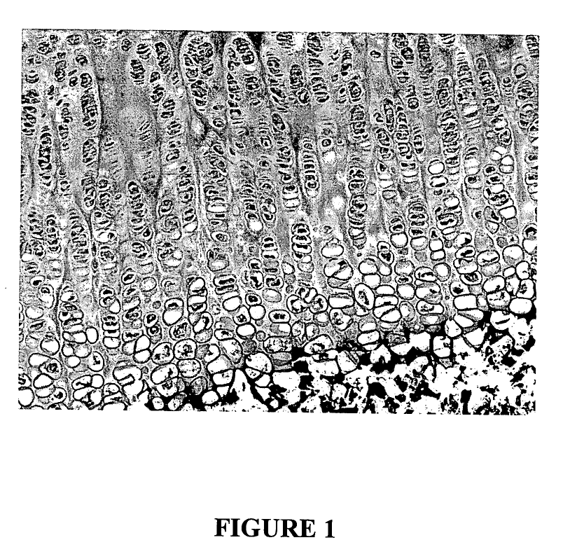

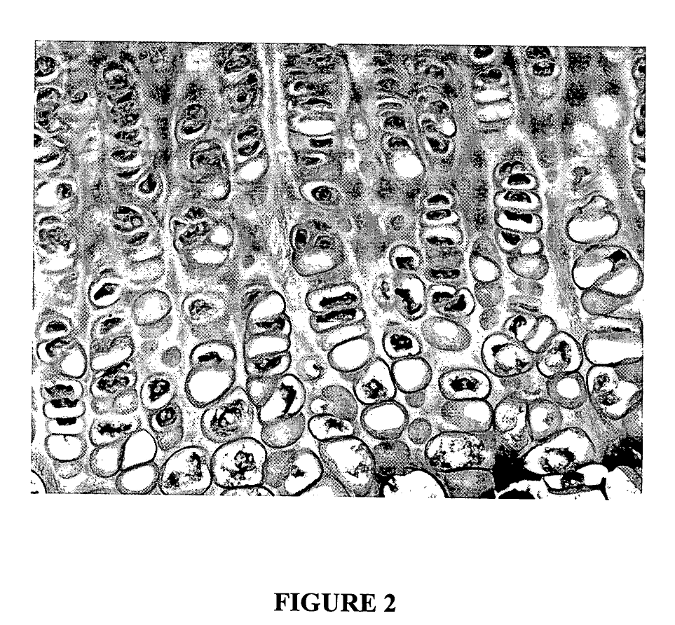

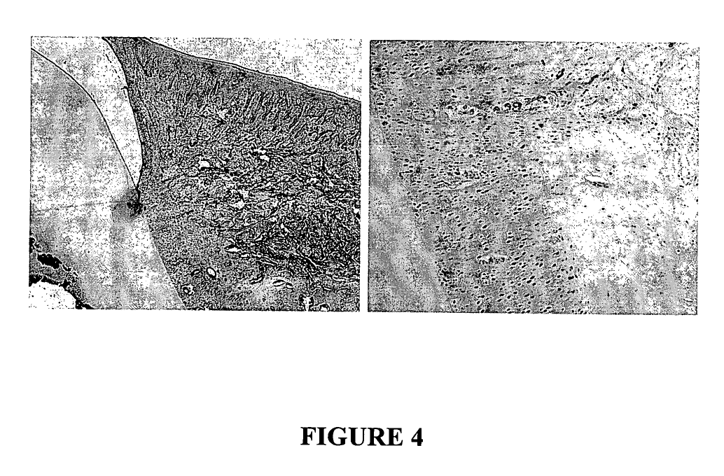

Additionally, dog growth plate tissues were obtained and stained for the relaxin receptor. Binding of relaxin to relaxin receptors was observed in chondrocytes of the growth plate with the most intense staining in the hypertrophic zone where cells are enlarging and storing glycogen (see FIGS. 1 and 2). This represents an area where cells are rapidly growing in size. Relaxin can aid in the initiation of development of chondrocytes, and can further aid in the division of bone cells and the production of bone. Other areas in bone (e.g., osteocytes in alveolar bone) that contained relaxin receptors were also identified, showing that relaxin targets a number of different sites (see FIG. 4).

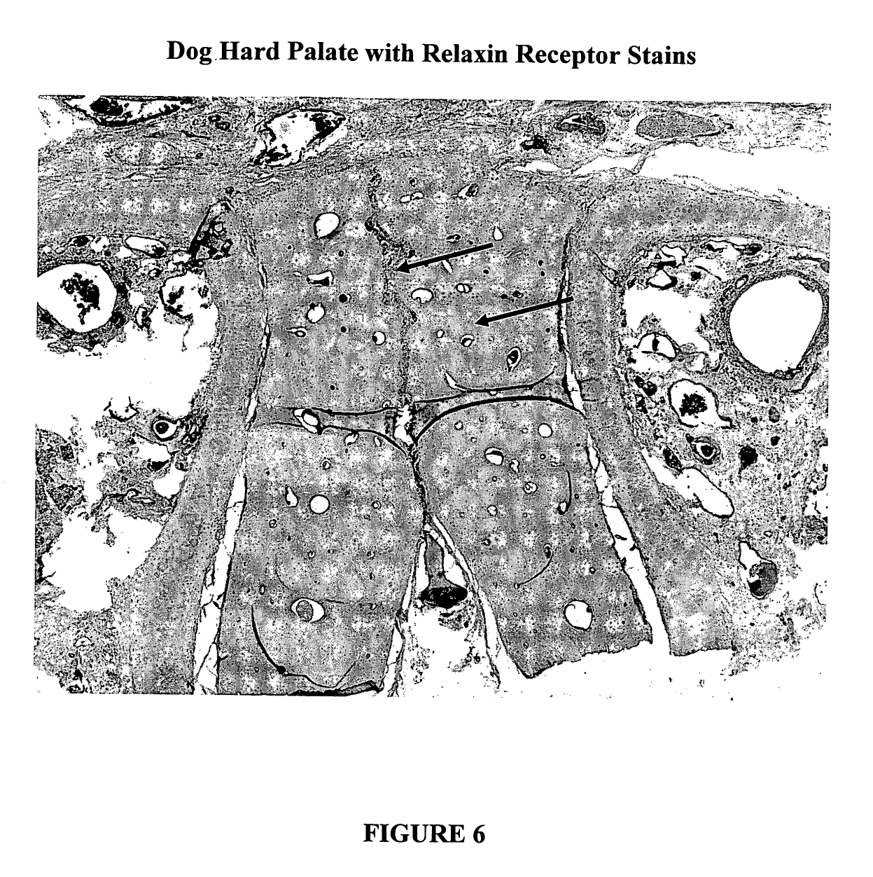

The hard palate of a dog is shown in FIG. 5. Relaxin receptors were also found in the dog hard palate (see FIGS. 6 and 7). The dog hard palate was stained (see FIG. 6) in order to visualize antibodies bound to relaxin receptors. The figures show that the antibodies to the relaxin receptors bound to cells in the sutural ligament, indicating the presence of relaxin receptors (see FIG. 7). Thus, relaxin can be employed to effect the sutural ligament and improve the expansion of the bone. Specifically, relaxin has the ability to soften ligamentous tissue by remodeling collagen, allowing the bones to be moved apart faster and with less pain. Relaxin's ability to soften the ligaments attached to these bones makes the separation of the bones easier.

As shown above, relaxin receptors were found in dog hard palate. Furthermore, relaxin receptors were found in periodontal ligament (PDL), gingival fibroblasts, gingival epithelium, osteocytes and odontoblasts in dog.

### Example 2

### Treating Inadequate Height

We found relaxin binding sites in chondrocytes (supra, see FIGS. 1 and 2) that are in an active stage of multiplying and storing glycogen. This has implications for relaxin binding involved in the growth of long bones (arms and legs) in adolescents. In fact, treatment of inadequate height in humans, particularly children and adolescents, can be achieved by administering relaxin compounds to the afflicted individual. The individual can be treated with relaxin compounds, either by systemic administration for general bone growth or by localized administration for specific bone growth.

### Example 3

### Treating Fractures

Bone fractures can be treated with relaxin compounds via localized or systemic administration. Notably, relaxin has an effect on fracture repair on multiple levels. Relaxin binds to the recruited osteoblasts and helps to stimulate new bone formation. Relaxin also possesses angiogenic properties in wound healing which are useful for treating bone fractures. Specifically, relaxin stimulates angiogenic factors (VEGF) from wound macrophages that are recruited to the fracture site. Hence, relaxin has a multilevel approach to fracture repair.

Various modifications and variations of the present invention will be apparent to those skilled in the art without departing from the scope and spirit of the invention. Although the invention has been described in connection with specific preferred embodiments, it should be understood that the invention as claimed should not be unduly limited to to such specific embodiments. Indeed, various modifications of the described modes for carrying out the invention which are obvious to those skilled in the art are intended to be within the scope of the claims. All publications, patents and patent applications cited in this specification are herein incoporated by reference in their entirety.

What is claimed is:

1. A method for modifying a target bone in a mammalian subject, said method comprising:
(i) administering a relaxin compound to said subject wherein said compound binds to a relaxin receptor in the target bone; and
(ii) monitoring a change in the target bone, wherein said change results in a modified target bone selected from the group consisting of remodeled bone, repaired bone, bone grown in size and bone grown in length.

2. The method of claim 1, wherein said modifying of the target bone is selected from the group consisting of growing, remodeling and repairing.

3. The method of claim 1, wherein said change is selected from the group consisting of bone growth, bone strength, bone movement, bone location, bone removal, bone deposition and bone fracture repair.

4. The method of claim 1, wherein said repaired bone is selected from the group consisting of a bone fracture, a bone malformation and a bone tumor.

5. The method of claim 1, wherein said target bone is any bone that comprises osteoblasts and osteoclasts.

6. The method of claim 1, wherein said relaxin compound is selected from the group consisting of H1 relaxin, H2 relaxin, H3 relaxin, relaxin-like factor (RLF) and combinations thereof.

7. The method of claim 6, wherein said H1 relaxin binds to a relaxin receptor selected from the group consisting of LGR7 and LGR8.

8. The method of claim 6, wherein said H2 relaxin binds to a relaxin receptor selected from the group consisting of LGR7 and LGR8.

9. The method of claim 6, wherein said H3 relaxin binds to a relaxin receptor selected from the group consisting of GPCR135 and GPCR142.

10. The method of claim 1, wherein said relaxin compound is a relaxin analog that functions as a receptor agonist.

11. The method of claim 10, wherein said receptor agonist is a compound that binds to said relaxin receptor in the target bone and activates said relaxin receptor.

12. The method of claim 1, wherein said relaxin compound is a relaxin analog that functions as a receptor antagonist.

13. The method of claim 12, wherein said receptor antagonist is a compound that binds to said relaxin receptor in the target bone and blocks said relaxin receptor.

14. The method of claim 1, wherein said relaxin receptor is found in bone tissue selected from the group consisting of osteoblasts, osteoclasts, osteocytes, odontoblasts and chondrocytes.

15. The method of claim 1, wherein said relaxin receptor is found in bone marrow tissue selected from the group consisting of mature myeloid elements of erythrocytes and mature myeloid elements of leukocytes.

16. The method of claim 1, wherein said relaxin receptor is found in bone marrow tissue of immature myeloid elements that are composed of hemocytoblasts, erythroblasts, myelocytes and megakaryocytes.

17. The method of claim 1, wherein said relaxin receptor is found in a sutural ligament.

18. The method of claim 1, wherein said mammalian subject is selected from the group consisting of human, dog, cat, rat, mouse and horse.

19. The method of claim 1, wherein said relaxin receptor is selected from the group consisting of LGR7, LGR8, GPCR135 and GPCR142.

20. A method for modifying height in a human subject, said method comprising:
(i) administering a relaxin compound to said human subject wherein said compound binds to a relaxin receptor in a target bone; and
(ii) monitoring said subject for a change in said height, wherein said target bone is selected from the group consisting of long bones of the limbs, skeletal bones and vertebral bones.

21. The method of claim 20, wherein said height is increased between about 0.2 to about 30 cm.

22. The method of claim 20, wherein said human subject is between about 8 and about 18 years of age.

23. The method of claim 20, wherein said human subject is a patient suffering from a condition selected from the group consisting of delayed growth, arthritis, Paget's disease, avascular necrosis, fibrous dysplasia, osteogenesis imperfecta (OI), primary hyperparathyroidism and growth hormone deficiency (GHD), growth hormone insensitivity syndrome (GHIS), and osteoporosis.
